(19) United States
(12) Patent Application Publication
(10) Pub. No.: US 2026/0345157 A1
(43) Pub. Date: Nov. 5, 2026

(54) SURGICAL STAPLING DEVICE WITH TISSUE GAP CONTROL

(71) Applicant: Covidien LP, Mansfield, MA (US)

(72) Inventors: John W. BEARDSLEY, Wallingford, CT (US); Stanislaw Z. MARCZYK, Stratford, CT (US); David A. NICHOLAS, Trumball, CT (US)

(21) Appl. No.: 19/123,506

(22) PCT Filed: Oct. 26, 2023
(86) PCT No.: PCT/US2023/077853

§ 371 Date: Apr. 23, 2025

Related U.S. Application Data
(60) Provisional application No. 63/420,370, filed on Oct. 28, 2022.

(51) Int. Cl. A61B 17/072

(52) U.S. Cl. CPC A61B 17/07207; A61B 2017/0725; A61B 2017/07271; A61B 2017/07285

(57) ABSTRACT

A surgical stapling device includes a cartridge assembly for accommodating tissues of different thicknesses. The cartridge assembly includes a channel member and a staple cartridge that is received in the channel member for movement between positions to change a size of a tissue gap defined between the staple cartridge and an anvil of the stapling device. The cartridge assembly also includes features formed on pushers or a cartridge body of the staple cartridge of the cartridge assembly to improve staple formation when stapling thin tissue.

### FIELD

This disclosure is directed to surgical stapling devices and, more particularly, to surgical stapling devices that include a tool assembly having a cartridge assembly for accommodating different tissue thicknesses.

### BACKGROUND

Surgical stapling devices for ejecting staples to join tissue or tissue segments in a fast and efficient manner in a variety of surgical procedures, e.g., anastomoses procedures, are well known. Typically, a surgical stapling device includes a tool assembly having first and second jaws that support a cartridge assembly and an anvil, respectively. The cartridge assembly includes a staple cartridge that supports a plurality of staples. The first and second jaws are mounted together to allow movement of the tool assembly between open and clamped positions. In the clamped position, the staple cartridge of the cartridge assembly and the anvil are supported in juxtaposed alignment and include tissue contact surfaces that define a predetermined tissue gap. The tissue gap and the size of the staples within the staple cartridge are dimensioned to receive and suture tissue having a thickness within a predetermined range to properly effect hemostasis. If the tissue positioned between the jaws is outside the predetermined range, i.e., either too thick or too thin, the staples may be malformed and not provide effective hemostasis. In addition, if the tissue thickness is misidentified by the clinician or if the tissue thickness falls near the outer edges of the range for a given staple size, the likelihood of ineffective hemostasis is increased.

A continuing need exists in the suturing arts for a surgical stapling device that can provide effective hemostasis for a greater range of tissue thicknesses.

### SUMMARY

This disclosure is directed to various aspects of a cartridge assembly of a stapling device for accommodating tissues of different thicknesses. In some aspects of the disclosure, the cartridge assembly includes a channel member and a staple cartridge that is received in the channel member for movement in relation to the channel member between positions to change a tissue gap defined between the staple cartridge and an anvil of the stapling device. Other aspects of the disclosure are directed to features formed on pushers or a cartridge body of the staple cartridge of the cartridge assembly to improve staple formation when stapling thin tissue.

One aspect of the disclosure is directed to a cartridge assembly including a channel member, a staple cartridge, and a spring. The channel member has a proximal portion and a distal portion and includes side walls and a bottom wall that define a cavity. The bottom wall defines a knife slot. The staple cartridge is received within the cavity of the channel member and includes a cartridge body, staples, pushers, and a staple guard. The cartridge body defines a knife slot and staple receiving pockets that are arranged in rows on each side of the knife slot. The staples and the pushers are received within the staple receiving pockets, and the pushers are movable from lowered positions to raised positions within the staple receiving pockets to eject the staples from the staple receiving pockets. The staple guard is supported on the cartridge body and is positioned to retain the staples and pushers within the cartridge body. The spring is secured to the bottom wall of the channel member in a pre-loaded state.

In aspects of the disclosure, the channel member includes a distal portion and a proximal portion, and the distal portion of the channel member defines an opening that extends through the bottom wall of the channel member.

In some aspects of the disclosure, the spring includes a wave spring that has a distal portion that includes a hook that is received in the opening of the channel member to secure the distal portion of the wave spring to the channel member.

In certain aspects of the disclosure, the wave spring includes a base portion and two elongate spring legs that extend distally from the base portion on opposite side of the knife slot, and the base portion supports the hook.

In aspects of the disclosure, each of the two elongate spring legs includes a proximal portion that is secured to the bottom wall of the channel member to secure the wave spring to the channel member in the preloaded state.

In some aspects of the disclosure, the proximal portion of each of the two elongate spring legs is welded to the bottom wall of the channel member.

In certain aspects of the disclosure, the channel member includes a finger that extends into the opening, and the hook is received about the finger.

In aspects of the disclosure, the base portion is formed separately from the two elongate spring legs, and the two elongate spring legs are secured to the base portion.

In some aspects of the disclosure, the opening is dimensioned to facilitate longitudinal movement of the hook within the opening when the wave spring is compressed beyond the pre-loaded state.

In certain aspects of the disclosure, the channel member includes a distal portion and a proximal portion, and the distal portion of the channel member defines spaced notches in opposite sides of the bottom wall of the channel member.

In aspects of the disclosure, the spring includes a wave spring that has a distal portion that includes two spaced hooks that are received within the spaced notches of the channel member to secure the distal portion of the wave spring to the channel member.

In some aspects of the disclosure, the hooks are longitudinally movable within the notches of the channel member when the wave spring is compressed beyond the pre-loaded state.

In certain aspects of the disclosure, each of the pushers includes at least one pusher plate, and each of the staples includes a back span and staple legs that extend from the back span.

In aspects of the disclosure, each of the pusher plates has an upper surface that defines a staple channel that receives the back span of the staple to support the staple on the pusher plate.

In some aspects of the disclosure, each of the pusher plates includes a radiused surface positioned on each side of the staple channel that is configured to direct the staple legs of a respective one of the staples outwardly of the back span as the staple is being formed.

In certain aspects of the disclosure, each of the pusher plates includes a chamfered surface positioned on each side of the staple channel that is configured to direct the staple legs of a respective one of the staples outwardly of the back span as the staple is being formed.

Other aspects of the disclosure are directed to a channel member having a body and a spring. The body has a proximal portion and a distal portion and includes side walls and a bottom wall that define a cavity. The bottom wall of the body of the channel member defines a knife slot. The spring is secured to the bottom wall of the channel member in a pre-loaded state.

Other aspects of the disclosure are directed to a cartridge assembly including a channel member, a staple cartridge, and a wave spring. The channel member has a proximal portion and a distal portion and includes side walls and a bottom wall that define a cavity. The bottom wall defines a knife slot. The staple cartridge is received within the cavity of the channel member and includes a cartridge body, staples, pushers, and a staple guard. The cartridge body defines a knife slot and staple receiving pockets arranged in rows on each side of the knife slot. The staples and the pushers are received within the staple receiving pockets, and the pushers are movable from lowered positions to raised positions within the staple receiving pockets to eject the staples from the staple receiving pockets. The staple guard is supported on the cartridge body and is positioned to retain the staples and pushers within the cartridge body. The wave spring is supported on the channel member between the bottom wall of the channel member and the staple guard of the staple cartridge. The wave spring includes a base portion and two spring legs that extend distally from the base portion along opposite sides of the knife slot. The two spring legs are secured to the bottom wall of the channel member in a pre-loaded state.

Still other aspects of the disclosure are directed to a cartridge assembly including a channel member and a staple cartridge. The channel member has a proximal portion and a distal portion and includes side walls and a bottom wall that define a cavity. The bottom wall defines a knife slot. The staple cartridge is received within the cavity of the channel member and includes a cartridge body, staples, pushers, and a staple guard. The cartridge body defines a knife slot and staple receiving pockets arranged in rows on each side of the knife slot. The staples and the pushers are received within the staple receiving pockets and the pushers are movable from lowered positions to raised positions within the staple receiving pockets to eject the staples from the staple receiving pockets. The staple guard is supported on the cartridge body and is positioned to retain the staples and pushers within the cartridge body. The staple guard includes a bottom wall that supports at least one spring member and defines a knife slot that is longitudinally aligned with the knife slot of the cartridge body. The at least one spring member is positioned to engage the bottom wall of the channel member to facilitate vertical movement of the bottom wall of the staple guard in relation to the bottom wall of the channel member.

In aspects of the disclosure, the at least one spring member includes at least one spring member positioned on each side of the knife slot of the staple guard.

In some aspects of the disclosure, the at least one spring member includes a plurality of spring members positioned on each side of the knife slot of the staple guard.

In certain aspects of the disclosure, each of the plurality of spring members on each side of the knife slot are longitudinally aligned with at least one other spring member of the plurality of spring members.

In aspects of the disclosure, each of the plurality of spring members on each side of the knife slot are longitudinally aligned with at least other two spring members of the plurality of spring members.

In some aspects of the disclosure, the bottom wall of the staple guard includes the at least one spring member.

In certain aspects of the disclosure, the at least one spring member is coupled to the bottom wall of the staple guard.

In aspects of the disclosure, the bottom wall of the staple guard defines openings and the at least one spring member includes proximal and distal extensions that are received in the openings in the bottom wall of the staple guard to couple the at least one spring member to the staple guard.

In some aspects of the disclosure, the at least one spring member is a wave spring.

Other aspects of the disclosure are directed to a tool assembly including an anvil and a cartridge assembly. The cartridge assembly is coupled to the anvil to facilitate pivotable movement of the tool assembly between an open position and a clamped position. The cartridge assembly includes a channel member and a staple cartridge. The channel member has a proximal portion and a distal portion and includes side walls and a bottom wall that define a cavity. The bottom wall defines a knife slot. The staple cartridge is received within the cavity of the channel member and includes a cartridge body, staples, pushers, and a staple guard. The cartridge body defines a knife slot and staple receiving pockets arranged in rows on each side of the knife slot. The staples and the pushers are received within the staple receiving pockets and the pushers are movable from lowered positions to raised positions within the staple receiving pockets to eject the staples from the staple receiving pockets. The staple guard is supported on the cartridge body and positioned to retain the staples and pushers within the cartridge body. The staple guard includes a bottom wall that supports at least one spring member and defines a knife slot that is longitudinally aligned with the knife slot of the cartridge body. The at least one spring member is positioned to engage the bottom wall of the channel member to facilitate vertical movement of the staple cartridge in relation to the anvil.

Still other aspects of the disclosure are directed to a staple cartridge that includes a cartridge body, staples, pushers, and a staple guard. The cartridge body defines a knife slot and staple receiving pockets arranged in rows on each side of the knife slot. The staples and the pushers are received within the staple receiving pockets and the pushers are movable from lowered positions to raised positions within the staple receiving pockets to eject the staples from the staple receiving pockets. The staple guard is supported on the cartridge body and is positioned to retain the staples and pushers within the cartridge body. The staple guard includes a bottom wall that defines a knife slot that is longitudinally aligned with the knife slot of the channel member. The bottom wall of the staple guard supports spring members positioned on opposite sides the knife slot of the staple guard.

Other aspects of the disclosure will be appreciated from the following description.

### BRIEF DESCRIPTION OF THE DRAWINGS

Various aspects of the disclosed stapling device are described herein below with reference to the drawings, wherein:

FIG. 1 is a side perspective view of a surgical stapling device including aspects of the disclosure with a tool assembly of the stapling device in an open position;

FIG. 2 is an enlarged view of the indicated area of detail shown in FIG. 1;

FIG. 3 is a side perspective view of a cartridge assembly of the tool assembly shown in FIG. 2;

FIG. 3A is an enlarged view of the indicated area of detail shown in FIG. 3;

FIG. 4 is a side perspective view of the cartridge assembly shown in FIG. 3 with a staple cartridge separated from a channel member of the cartridge assembly;

FIG. 5 is an exploded view of the cartridge assembly shown in FIG. 4;

FIG. 5A is a side view of the tool assembly of the stapling device 10 with thin tissue clamped between the anvil and the cartridge assembly;

FIG. 5B is a side view of the tool assembly of the stapling device 10 with thick tissue clamped between the anvil and the cartridge assembly,

FIG. 5C is a side view of the tool assembly of the stapling device 10 with tissue having a non-uniform thickness clamped between the anvil and the cartridge assembly;

FIG. 5D is a cross-sectional view taken along section line 5D-5D of FIG. 5C;

FIG. 6 is a perspective view from above of the channel member and wave spring of the cartridge assembly shown in FIG. 5 with the wave spring secured to the channel member,

FIG. 7 is a side perspective view of an alternate version of the channel member and wave spring of the cartridge assembly shown in FIG. 5 with parts separated;

FIG. 8 is perspective view from above of the channel member and wave spring shown in FIG. 7 with the wave spring secured to the channel member;

FIG. 9 is a cross-sectional view taken along section line 9-9 of FIG. 8;

FIG. 10 is a cross-sectional view taken along a longitudinal axis of the channel member as the wave spring is secured to the channel member;

FIG. 11 is a cross-sectional view of the channel member and wave spring shown in FIG. 10 with a staple cartridge loaded into the channel member;

FIG. 12 is an enlarged view of the indicated area of detail shown in FIG. 11;

FIG. 13 is side cross-sectional view taken along the longitudinal axis if the cartridge assembly as the stapling device shown in FIG. 1 is fired;

FIG. 14 is a side perspective view of another alternate version of the channel member and wave spring of the cartridge assembly shown in FIG. 5 with parts separated;

FIG. 15 is an enlarged perspective view from below of a distal portion of the channel member and wave spring shown in FIG. 14 assembled;

FIG. 16 is a side perspective view from above of a distal portion of another alternate version of the wave spring of the cartridge assembly shown in FIG. 5;

FIG. 17 is a side view of the distal portion of the wave spring shown in FIG. 16;

FIG. 18 is a side perspective view from above of the wave spring shown in FIG. 16 assembled to the channel member;

FIG. 19 is a side perspective view from above of another alternate version of the channel member and wave spring of the cartridge assembly shown in FIG. 5;

FIG. 20 is a perspective view from above of a distal portion of the wave spring shown in FIG. 19;

FIG. 21 is a bottom perspective view of an alternate version of the staple cartridge of the cartridge assembly shown in FIG. 4;

FIG. 22 is a side view of the staple cartridge shown in FIG. 21;

FIG. 23 is a bottom perspective view of another alternate version of the staple cartridge of the cartridge assembly shown in FIG. 4;

FIG. 24 is a bottom perspective view of the staple cartridge of the cartridge assembly shown in FIG. 23 with the wave spring separated from a staple guard of the staple cartridge;

FIG. 25 is a side perspective view of a distal portion of another version of the cartridge assembly shown in FIG. 3;

FIG. 26 is an enlarged view of the indicated area of detail shown in FIG. 25;

FIG. 27 is a perspective view from the distal end of a pusher and staple of the cartridge assembly shown in FIG. 25 after the staple is formed;

FIG. 28 is a cross-sectional view taken along section line 28-28 of FIG. 27;

FIG. 29 is a perspective view from the distal end of an alternate version of the pusher and staple of the cartridge assembly shown in FIG. 25 after the staple is formed;

FIG. 30 is a cross-sectional view taken along section line 30-30 of FIG. 29;

FIG. 31 is a side perspective view of a portion of an alternate version of a cartridge body of the staple cartridge shown in FIG. 4;

FIG. 32 is a perspective view from above of the cartridge body shown in FIG. 31 as a staple is formed; and

FIG. 33 is an alternate version of the cartridge body of the staple cartridge shown in FIG. 4.

### DETAILED DESCRIPTION

The disclosed surgical stapling device will now be described in detail with reference to the drawings in which like reference numerals designate identical or corresponding elements in each of the several views. However, it is to be understood that the disclosed aspects are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

In this description, the term “proximal” is used generally to refer to that portion of the device that is closer to a clinician when the device is used in its customary fashion, while the term “distal” is used generally to refer to that portion of the device that is farther from the clinician when the device is used in its customary fashion. In addition, the term “clinician” is used generally to refer to medical personnel including doctors, nurses, and support personnel. Further, directional terms such as front, rear, upper, lower, top, bottom, and similar terms are used to assist in understanding the description and are not intended to limit the disclosure.

Surgical stapling devices are used in a variety of surgical procedures to treat tissues having a variety of thicknesses. Generally during a surgical procedure, a surgeon selects a staple size to be used during the surgical procedure based on a variety of factors such as the tissue to be treated, the type of procedure being performed, personal preference, and personal experience. The inventors have recognized, inter alia, that a problem to be solved can include surgeons selecting stapling devices having a staple size that does not provide effective hemostasis. The present disclosure can help provide a solution to this problem such as by providing a cartridge assembly of the stapling device that can accommodate tissues of different thicknesses. Providing a cartridge assembly that can accommodate a large range of tissue thickness can make staple size selection simpler, reduce surgeon error, and, thus, improve clinical outcomes. Additionally, since fewer options are needed to cover a range of targeted tissue thickness, this disclosure simplifies logistics and offers savings for hospitals as the hospitals do not need to have a large number of options in stock.

The disclosed surgical stapling device includes a tool assembly having a cartridge assembly for accommodating tissues of different thicknesses. The cartridge assembly includes a channel member and a staple cartridge that is received in the channel member for movement in relation to the channel member between positions to change a tissue gap defined between the staple cartridge and an anvil of the stapling device. The cartridge assembly may also include features formed on pushers or a cartridge body of the staple cartridge of the cartridge assembly to improve staple formation when stapling thin tissue.

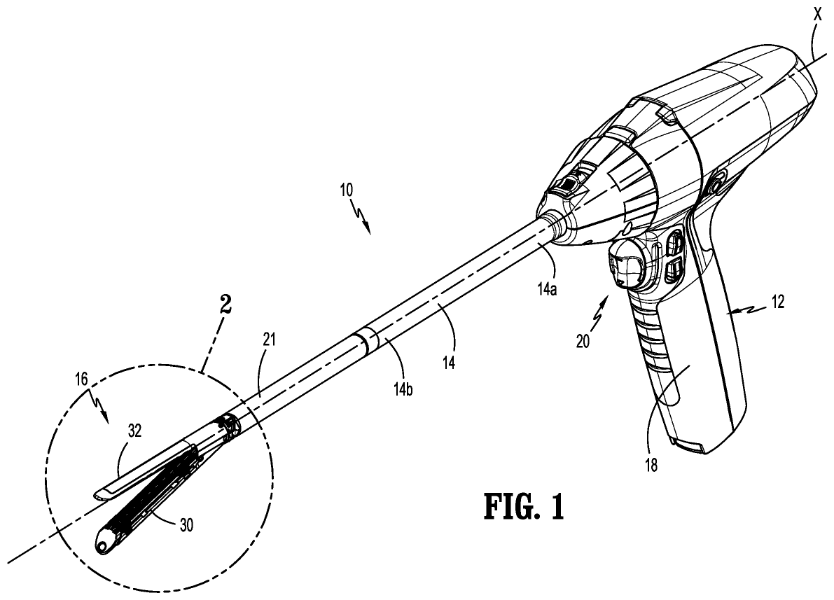

FIG. 1 illustrates a surgical stapling device shown generally as stapling device 10 that includes a handle assembly 12, an elongate body or adapter assembly 14, and a tool assembly 16. The handle assembly 12 is powered and includes a stationary handgrip 18 and one or more actuation buttons 20. The actuation buttons 20 are operable to actuate various functions of the tool assembly 16 via the adapter assembly 14 including approximation, stapling, and cutting. In certain aspects of the disclosure, the handle assembly 12 supports batteries (not shown) that provide power to the handle assembly 12 to operate the stapling device 10. Although the stapling device 10 is illustrated as a powered stapling device, it is envisioned that the disclosed tool assembly 16 is suitable for use with manually powered surgical stapling devices as well as robotically controlled stapling devices.

The adapter assembly 14 includes a proximal portion 14a and a distal portion 14b. The proximal portion 14a is coupled to the handle assembly 12 and the distal portion 14b supports the tool assembly 16. In aspects of the disclosure, the tool assembly 16 forms part of a reload assembly 21 that is removably supported on the distal portion 14b of the adapter assembly 14 and can be replaced after the stapling device 10 is fired to facilitate reuse of the stapling device 10. It is also envisioned that the tool assembly 16 can be fixedly coupled to the distal portion 14b of the adapter assembly 14.

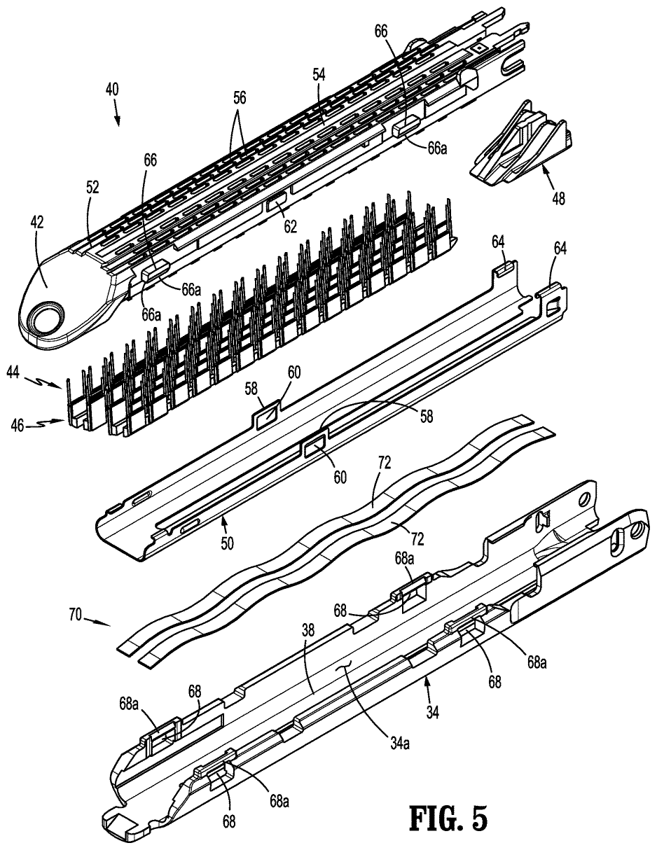

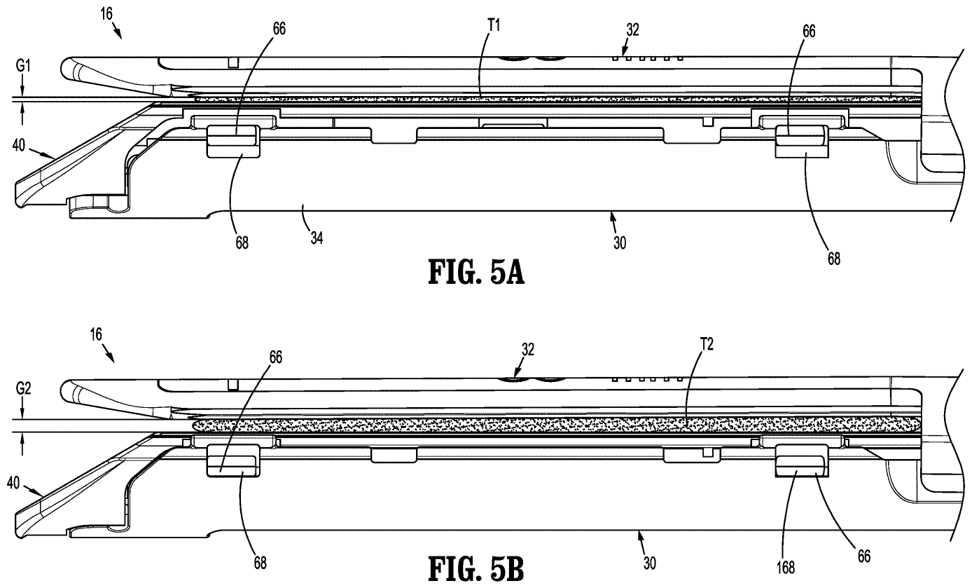

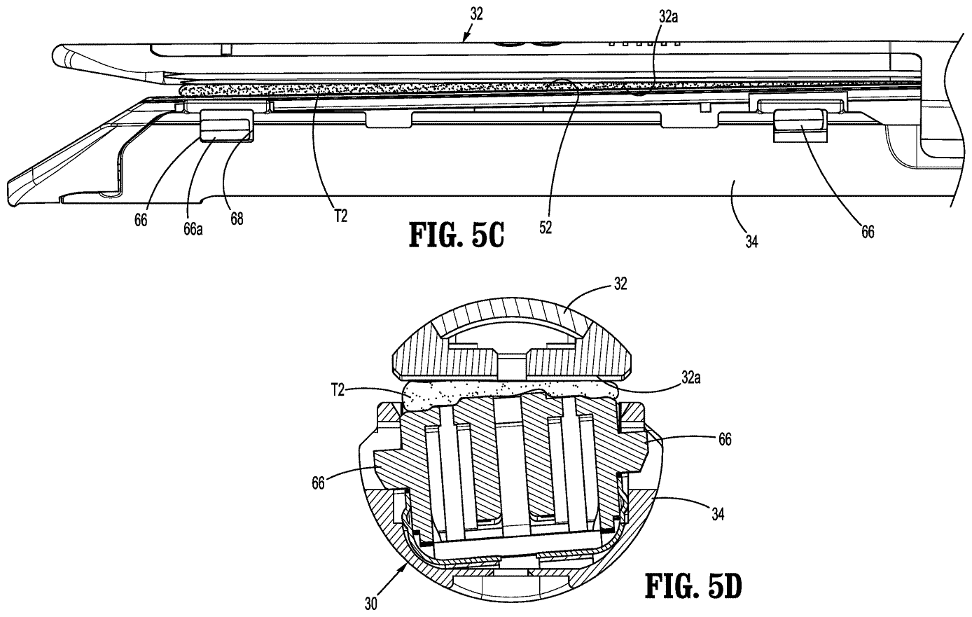

The tool assembly 16 of the stapling device 10 includes a cartridge assembly 30 and an anvil 32. The cartridge assembly 30 and the anvil 32 are coupled together such that the tool assembly 16 can pivot between an open position (FIG. 1) and a clamped position. In the clamped position, the anvil 32 and the cartridge assembly 30 are in juxtaposed alignment with each other to define a tissue gap for receiving tissue. The anvil 32 includes a tissue engaging surface 32a (FIG. 5A) that defines staple forming pockets (not shown).

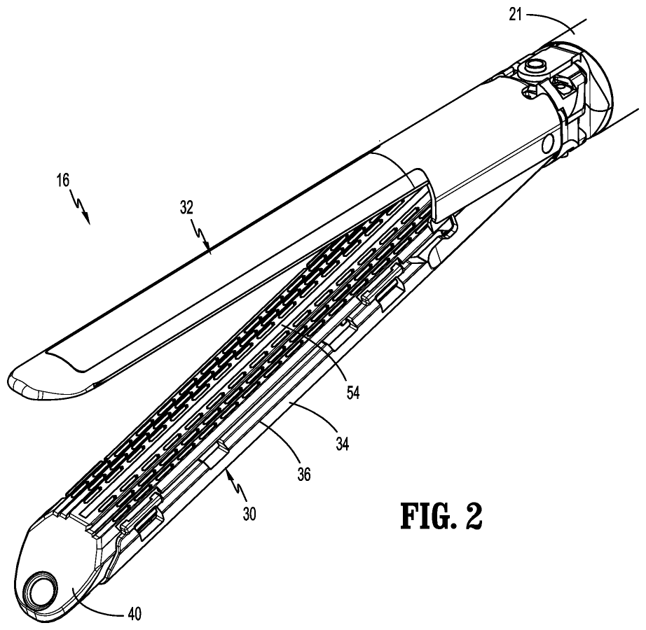

FIGS. 2-5 illustrate the cartridge assembly 30 which includes a channel member 34 and a staple cartridge 40. The channel member 34 includes side walls 36 and a bottom wall 38 (FIG. 4) that define a cavity 34a (FIG. 5) that receives the staple cartridge 40. The bottom wall 38 defines a knife slot 38a. In aspects of the disclosure, the staple cartridge 40 can be removably received within the cavity 34a of the channel member 34 to allow for replacement of the staple cartridge 40 after each firing of the stapling device 10 (FIG. 1) to facilitate reuse of the stapling device 10 (FIG. 1).

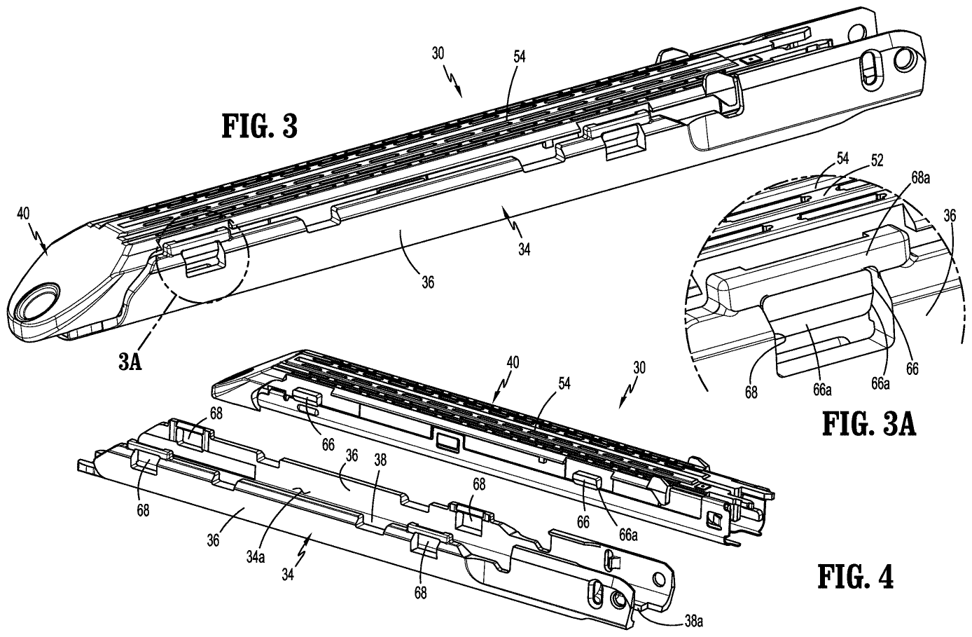

FIG. 5 illustrates an exploded view of the staple cartridge 40 which includes a cartridge body 42, staples 44, pushers 46, an actuation sled 48, and a staple guard 50. The cartridge body 42 of the staple cartridge 40 is received within the cavity 34a defined by the channel member 34 and includes a tissue engaging surface 52. The cartridge body 42 defines a knife slot 54 and a plurality of staple receiving pockets 56 that are arranged in rows on each side of the knife slot 54. The knife slot 54 and the staple receiving pockets 56 open onto the tissue engaging surface 52 of the cartridge body 42. In certain aspects of the disclosure, the tissue engaging surface 52 of the staple cartridge 40 has a stepped configuration with a raised surface adjacent the central knife slot 54 and lower surfaces positioned outwardly of the central knife slot 54 towards the side walls 36 of the channel member 34. Each of the staple receiving pockets 56 receives a staple 44 (FIG. 3) and at least a portion of one of the pushers 46. In some aspects of the disclosure, the staples 44 in the inner rows of staple receiving pockets 56 are shorter in length than the staples 46 in the outer rows of staple receiving pockets 56. The actuation sled 48 is movable distally from a proximal end of the cartridge body 42 into sequential engagement with the pushers 46 to urge the pushers 46 upwardly as viewed in FIG. 5 to eject the staples 44 from the staple receiving pockets 56 of the cartridge body 42 through the tissue engaging surface 52 and into engagement with the anvil 32 (FIG. 1).

The staple guard 50 is secured to a bottom side of the cartridge body 42 to retain the staples 44, the pushers 46, and the actuation sled 48 within the cartridge body 42. In aspects of the disclosure, the staple guard 50 includes flexible links 58 that define openings 60 that receive protrusions 62 formed on an outer surface of the cartridge body 42 to secure the staple guard 50 to the cartridge body 42. In some aspects of the disclosure, the staple guard 50 also includes hooked fingers 64 that engage a proximal portion of the cartridge body 42 to secure the staple guard 50 to the cartridge body 42. U.S. Pat. No. 6,241,139 discloses exemplary aspects of the construction and operation of a staple cartridge of a surgical stapling device.

The cartridge body 42 of the staple cartridge 40 includes tabs 66 and the channel member defines windows 68 that receive the tabs 66. The windows 68 are dimensioned to allow the tabs 66 of the cartridge body 42 to move vertically within the windows 68 but prevent any substantial longitudinal movement of the staple cartridge 40 within the cavity 34a of the channel member 34. In aspects of the disclosure, the upper ends of the windows 68 are enclosed by bars 68a that prevent the tabs 66 from exiting the windows 68. In some aspects of the disclosure, the tabs 66 can have convex wall portions 66a on opposite ends of the tabs 66 and/or on the side walls of the tabs 66. The convex wall portions 66a allow the cartridge body 42 to pitch (FIG. 5C) and/or roll (FIG. 5D) within the cavity 34a of the channel member 34 to account for differences in tissue thickness along the staple line. It is noted that tabs 66 need not have convex wall portions to accommodate pitching and rolling of the cartridge body 42 within the cavity 34a of the channel member 34. Alternately, the windows 68 and tabs 66 can be dimensioned to facilitate pitching and rolling of the cartridge body 42. Thus, even though the staples 44 are the same size along each of the rows of staples 44, the movement of the cartridge body 42 within the cavity 34a of the channel member 34 allows for the formed configuration of the staples to be different along the staple lines in a single firing of the stapling device 10.

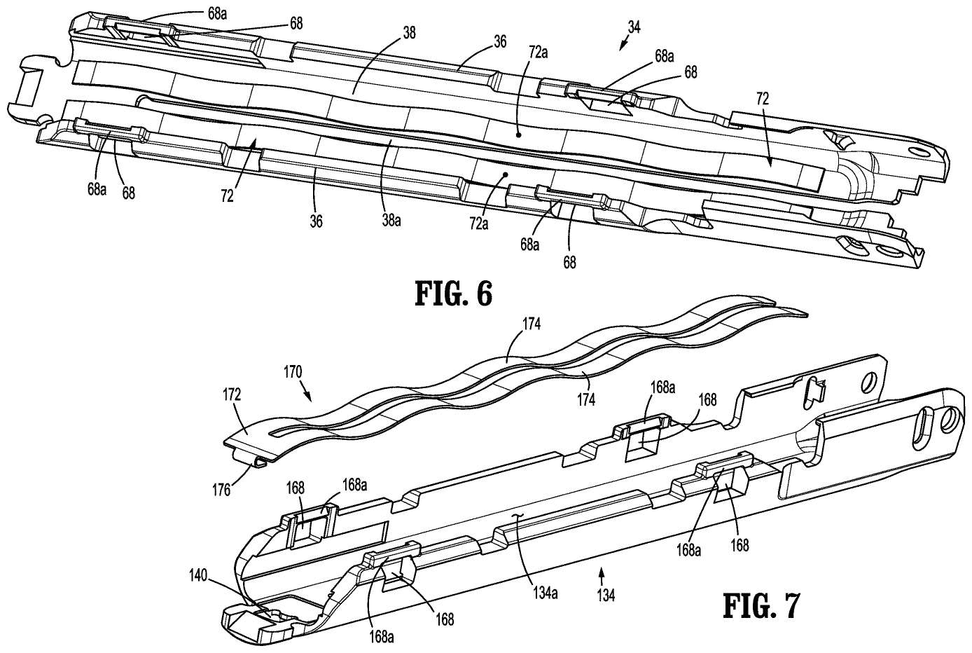

The cartridge assembly 30 includes one or more elongate springs or biasing members 70 positioned between the staple guard 50 and the bottom wall 38 of the channel member 34. The biasing member(s) urges the staple cartridge 40 upwardly within the cavity 34a of the channel member 34 towards the anvil 32. In aspects of the disclosure, the biasing members 70 can be elongated wave springs 72 that are positioned on opposite sides of the knife slot 38a between the staple guard 50 of the staple cartridge 40 and the bottom wall 38 of the channel member 34 and have sinusoidal configurations along the longitudinal axis of the channel member 34. In aspects of the disclosure, the wave springs 72 can be spot welded to the channel member 34 and are compressible to allow vertical movement of the staple cartridge 40 within the cavity 34a of the channel member 34. In some aspects of the disclosure, the spot welds 72a (FIG. 6) are positioned on a central portion of the wave springs 70 and a central portion of the channel member 34. However, the wave springs 72 can be spot welded or secured to the channel member 34 using other fastening techniques at any position along the length of the wave spring 72 that allows the wave spring 72 to compress such that the cartridge body 42 can move vertically within the channel member 34.

When tissue is positioned between the anvil 32 and the cartridge assembly 30 and the tool assembly 16 is moved from the open position to the clamped position, the force of the tissue on the cartridge body 42 of the staple cartridge 40 compresses the biasing member 70 to move the staple cartridge 40 vertically (or towards the bottom wall 38 of the channel member 34) within the cavity 34a of the channel member 34. The amount of movement of the staple cartridge 40 within the cavity 34a of the channel member 34 during clamping and firing of the stapling device 10 (FIG. 1) will depend on the thickness of the tissue. More specifically, the staple cartridge 40 will move downwardly within the cavity 34a of the channel member 34 a greater distance as the tissue thickness increases. As the staple cartridge 40 moves within the channel member 34, the tabs 66 move within the windows 68 defined in the side walls 36 of the channel member 34. As described above, the tabs 66 and the windows 68 allow the staple cartridge 40 to move vertically within the cavity 34a defined by the channel member 34 but prevent longitudinal movement of the staple cartridge 40 within the cavity 34a. As the staple cartridge 40 moves within the channel member 34, the relative position of the pushers 46 change in relation to the anvil 32 which affects staple formation. More specifically, when thick tissue is clamped, the wave springs 72 supporting the staple cartridge 40 compress to allow the cartridge body 42 to move within the cavity 34a of the channel member 34 to define a first tissue gap G2 (FIG. 5B) between the tissue engaging surface 52 of the cartridge body 42 and the tissue engaging surface 32a of the anvil 32, and when thinner tissue is clamped, the wave springs 72 supporting the staple cartridge 40 compress to allow the cartridge body 42 to move within the cavity 34a of the channel member 34 to define a second tissue gap G1 (FIG. 5A) between the tissue engaging surface 52 of the cartridge body 42 and the tissue engaging surface 32a of the anvil 32, wherein the first tissue gap G2 is greater than the second tissue gap G1.

The staples 44 in the inner, middle, and outer rows of staples 44 can have the same unformed size. Alternately, the staples 44 in the inner, middle, and outer rows of staples 44 can have different unformed sizes in each of the rows of staples. For example, the staples 44 in the inner rows of staples 44 can have a smaller unformed size than the staples in the middle and and/or outer rows of staples 44 in addition, the tissue engaging surface 52 (FIG. 5A) of the cartridge body 42 of the staple cartridge 40 can be planar or, alternately, the tissue engaging surface 52 can be stepped.

It is noted that the staples 44, although having the same unformed sizes in a particular row of staples 44, may have different formed sizes. This difference results from the movement of the cartridge body 42 within the cavity 34a of the channel member 34 caused by a variation in tissue thickness along the length of the staple line. As described above, the cartridge body 42 can rock back and forth within the cavity 34a of the channel member 34. This may occur when tissue in the distal portion of the tool assembly 16 is thicker than tissue in the proximal portion of the tool assembly 16 such that the tissue gap in the distal portion of the cartridge body 42 is greater than the tissue gap in the proximal portion of the cartridge body 42.

FIGS. 7-9 illustrate an alternate version of the channel member and the wave spring of the cartridge assembly 30 (FIG. 3) shown generally as channel member 134 and wave spring 170. The channel member 134 is like the channel member 34 (FIG. 5) and includes side walls 136 and a bottom wall 138 that together define a cavity 134a that receives the staple cartridge 40 (FIG. 4). The bottom wall 138 of the channel member defines a knife slot 138a and an opening 140 that extends through the bottom wall 138 of the channel member 134 and communicates with the knife slot 138a.

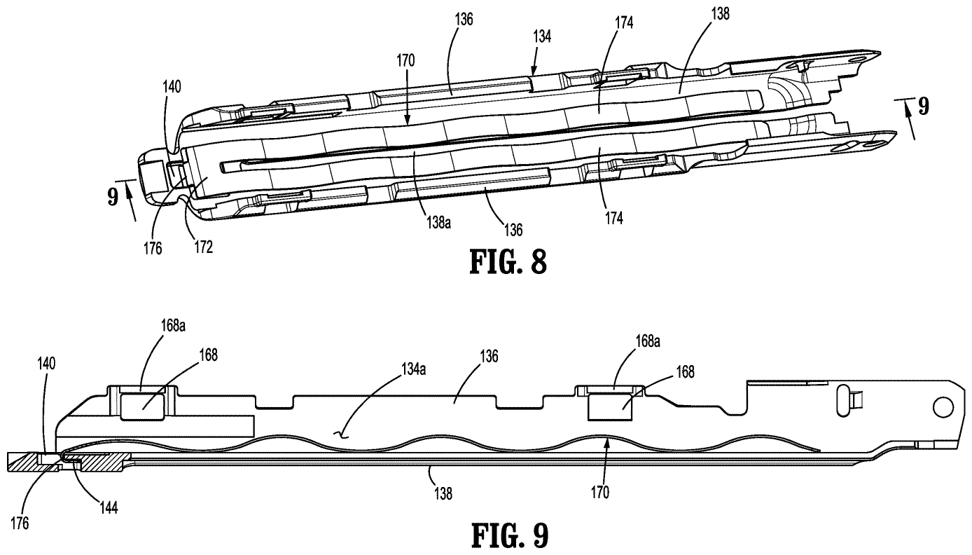

The wave spring 170 includes a base portion 172 and two elongate spring legs 174 that extend from the base portion 170 and are positioned along opposite sides of a knife slot 138a (FIG. 8). The base portion 172 of the wave spring 170 supports or includes a hook 176. The wave spring 170 is secured to the bottom wall 138 of the channel member 134 by positioning the hook 176 of the wave spring 170 through the opening 140. More specifically, as illustrated in FIGS. 8 and 9, the hook 176 is positioned through the opening 140 and engages the outer surface of the channel member 134 to secure the distal end of the wave spring 170 to the channel member 134. The opening 140 in the channel member 134 has a length that is greater than the length of the hook 176 of the wave spring 170 to facilitate longitudinal movement of the hook 176 within the opening 140. In aspects of the disclosure, the bottom wall 138 of the channel member 134 includes a finger 144 (FIG. 9) that extends partly across the opening 140, and the hook 176 is received about the finger 144 to secure the distal portion of the wave spring 170 to the distal portion of the channel member 134.

It is envisioned that other fastening structure or techniques that allow for spring compression within the cavity 134a of the channel member 134 could be used in place of the hook 176 and opening 140 to secure the wave spring 170 to the channel member 134. For example, the wave spring may include a pin (not shown) and the channel member 134 may define a slot (not shown) that receives the pin.

When the distal portion of the wave spring 170 is secured to the distal portion of the channel member 134, the two elongate spring legs 174 of the wave spring 170 extend proximally from the base portion 172 of the wave spring 170 along opposite sides of the knife slot 138a. Each of the spring legs 174 of the wave spring 170 has a sinusoidal configuration and is compressible to facilitate vertical movement of a staple cartridge 40 within the cavity 134a of the channel member 134.

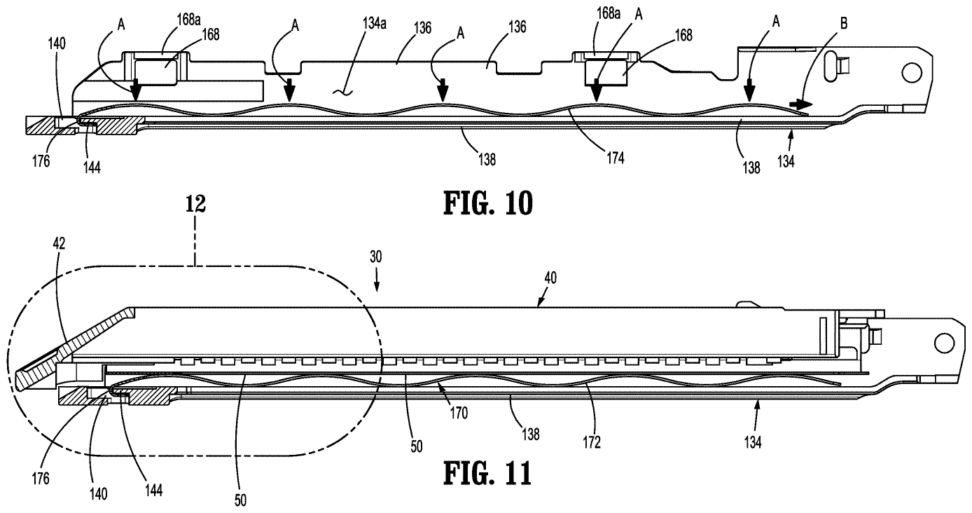

FIG. 10 illustrates the wave spring 170 as the wave spring 170 is secured to the channel member 134 during manufacture of the cartridge assembly 30. In aspects of the disclosure, when the wave spring 170 is secured to the bottom wall 138 of the channel member 134, the wave spring 170 is pre-loaded to facilitate placement of the staple cartridge 40 into the cavity 134a of the channel member 134 by a clinician. In aspects of the disclosure, the wave spring 170 is pre-loaded into the cavity 134a of the channel member 134 during the manufacturing process by inserting the hook 176 into the opening 140 in the channel member 134 to receive the finger 144, subsequently compressing the wave spring 170 in the direction of arrows “A”, and finally welding proximal portions of the spring legs 174 to the bottom wall 138 of the channel member 134 while the spring legs 174 are partially compressed or pre-loaded. As illustrated, the distal portion of the wave spring 170 will move in the direction of arrow “B” when the wave spring 170 is pre-loaded. It is envisioned that other portions of the wave spring 170 can be welded or secured to the channel member 134 at locations other than the proximal portions of the spring legs 174, e.g., central portions of the wave spring 170.

In aspects of the disclosure, the wave spring 170 is secured to the bottom wall of the channel member 134 in a pre-loaded state to allow vertical movement of the staple cartridge 40 within the cavity 134a of the channel member 134. In one aspect of the disclosure, the staple cartridge 40 moves vertically from about 0.015 inch (0.381 millimeter (mm)) to about 0.025 inch (0.635 millimeter (mm)) within the cavity 134a of the channel member 134. In certain aspects of the disclosure, the wave spring 170 is secured to the bottom wall 138 of the channel member 134 in a pre-loaded state to allow for about 0.020 inch (0.0508 millimeter (mm)) of vertical movement of the staple cartridge 40 (FIG. 4) within the cavity 134a of the channel member 134. Alternately, the wave spring 170 and channel member 134can be configured to provide a greater range of motion of the staple cartridge 40 within the channel member 134.

Preloading of the wave spring 170 can be accomplished during the manufacturing process by positioning the channel member 134 and the wave spring 170 within a fixture (not shown), pre-compressing the wave spring 170, and subsequently spot welding or securing the proximal portions of the spring legs 174 to the channel member 134 using some other fastening technique.

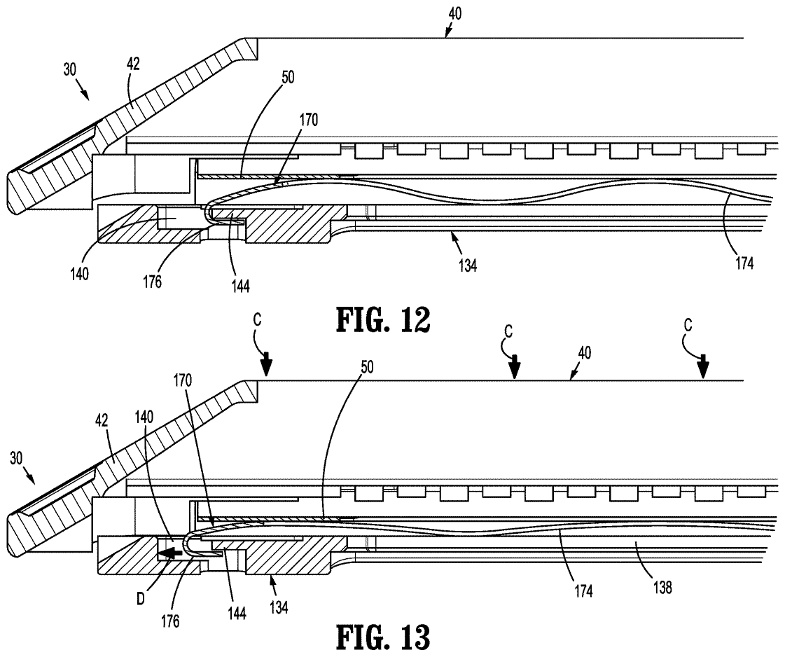

FIGS. 11 and 12 illustrate the staple cartridge 40 (FIG. 5) as the staple cartridge 40 is loaded into the cavity 134a of the channel member 134 with the wave spring 170 in a preloaded state within the cavity 134a of the channel member 134. As the staple cartridge 40 is loaded into the cavity 134a of the channel member 134, the tabs 66 on the cartridge body 42 (FIG. 5) of the staple cartridge 40 (FIG. 5) are received within the windows 168 of the channel member 134. When the staple cartridge 40 is received into the cavity 134a of the channel member 134, the guard 50 is seated on the wave spring 170 and the tabs 66 (FIG. 5A) of the cartridge body 42 are positioned in the upper ends of the windows 168. When the tool assembly 16 is moved to the clamped position about thin tissue “T1”, the tabs 66 will be positioned in the upper ends of the windows 168, and the tissue gap “G1” (FIG. 5A) defined between the staple cartridge 40 of the cartridge assembly and the anvil 32 will be at its smallest distance. When the tool assembly 16 is moved to the clamped position about thick tissue “T2”, the tabs 66 will be positioned in the lower ends of the windows 168, and the tissue gap “G2” (FIG. 5B) defined between the staple cartridge 40 of the cartridge assembly and the anvil 32 will be at its largest distance. As described, the distance from the bottom surface of the tab 66 and the bottom surface of the window 68 is the maximum distance the cartridge body 42 can move within the cavity 34a of the channel member 34. It is noted that pre-compressing the wave spring 170 allows the staple cartridge 40 to be inserted into the cavity 134a of the channel member 134 with minimal force.

FIG. 13 illustrates the cartridge assembly 30 (FIG. 3) including the staple cartridge 40 and the channel member 134 as the stapling device 10 (FIG. 1) is fired. As the stapling device 10 is fired, and a clamping member (not shown) is driven through the tool assembly 16, forces applied by tissue to the tissue engaging surface 52 of the cartridge body 42 of the staple cartridge 40 moves the staple cartridge 40 downwardly in the direction of arrows “C” within the cavity 134a of the channel member 134 to accommodate thicker tissue. The degree of movement of the staple cartridge 40 within the cavity 134a of the channel member 134 will be proportional to the thickness of the tissue that is compressed between the staple cartridge 40 and the anvil 32 (FIG. 1). As described above, proximal portions of the spring legs 174 of the wave spring 170 are secured to the bottom wall 138 of the channel member 134. Thus, when the staple cartridge 40 moves within the cavity 134a of the channel member 134 during firing to compress the spring 134 further from the pre-loaded state, the wave spring 170 flattens such that the distal end of the wave spring 170 moves distally in the direction of the arrow “D” within the opening 140 in the channel member 134. The hook 176 of the wave spring 170 and the finger 144 of the channel member 134 should be dimensioned to prevent separation of the hook 176 from the finger 144 during maximum compression of the wave spring 170.

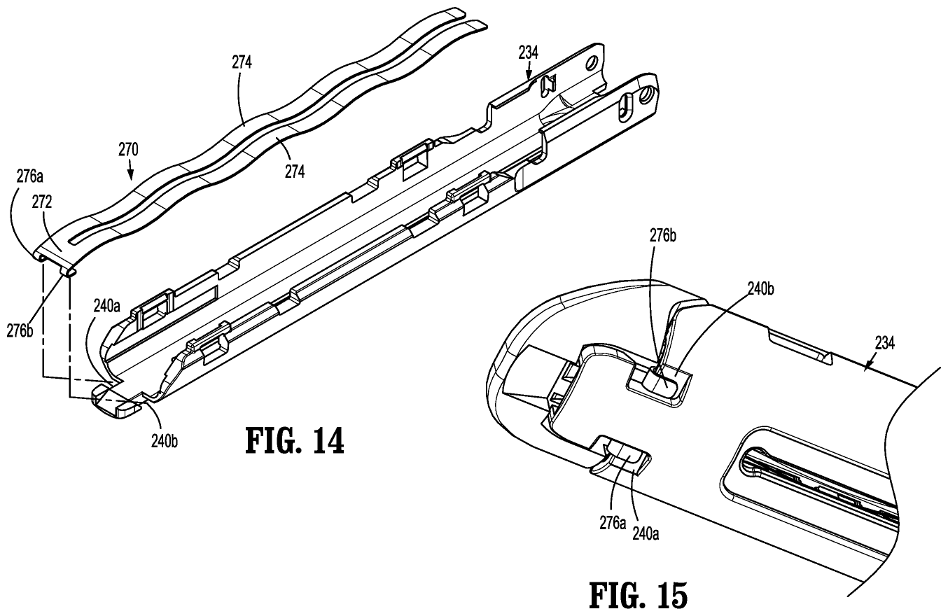

FIGS. 14 and 15 illustrate an alternate version of the channel member and wave spring of the cartridge assembly 30 (FIG. 3) shown generally as channel member 234 and wave spring 270. The channel member 234 is like the channel members 34, 134 described above except that the opening 140 (FIG. 7) of the channel member 134 is replaced with two spaced apart notches 240a, 240b. The wave spring 270 is like the wave spring 170 (FIG. 7) except that the hook 176 is replaced by two spaced hooks 276a, 276b that are supported on a base portion 272 of the wave spring 270. Each of the hooks 276a, 276b is received in one of the notches 240a, 240b of the channel member 234 and the spring legs 274 of the wave spring 270 extend proximally from the base portion 272. Providing two spaced apart notches 240a, 240b and two hooks 276a, 276b as compared to a single larger opening and one large hook allows the structural integrity of the channel member 234 to be better maintained. The wave spring 270 can be secured to the bottom wall 238 of the channel member 234 in a pre-loaded state as described above regarding wave spring 170.

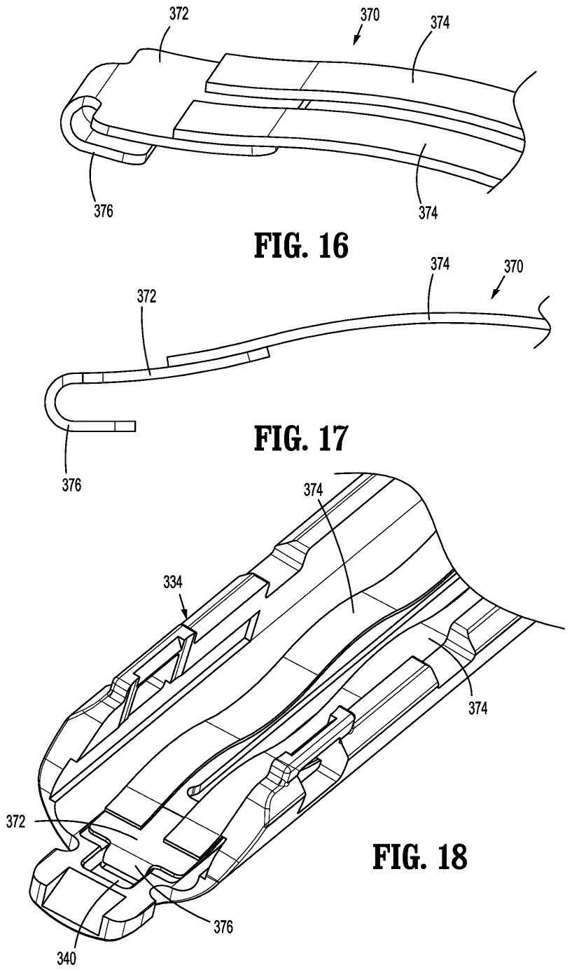

FIGS. 16-18 illustrate another alternate version of the channel member and wave spring shown generally as channel member 334 (FIG. 18) and wave spring 370. The channel member 334 is like the channel member 134 (FIG. 8) and includes an opening 340 (FIG. 18). The wave spring 370 is like the spring 170 (FIG. 7) except that the base portion 372 and hook 376 are formed separately from each of the spring legs 374, and the spring legs 374 are joined to the base portion 372 such as by welding or crimping. The hook 376 of the wave spring 370 is secured to the channel member 334 as described above regarding wave spring 170 and channel member 134. As illustrated in FIG. 18, the hook 376 can move within the opening 340 when the wave spring 370 is compressed. The wave spring 370 can be secured in a pre-loaded state to the bottom wall 338 of the channel member 334 as described above regarding wave spring 170.

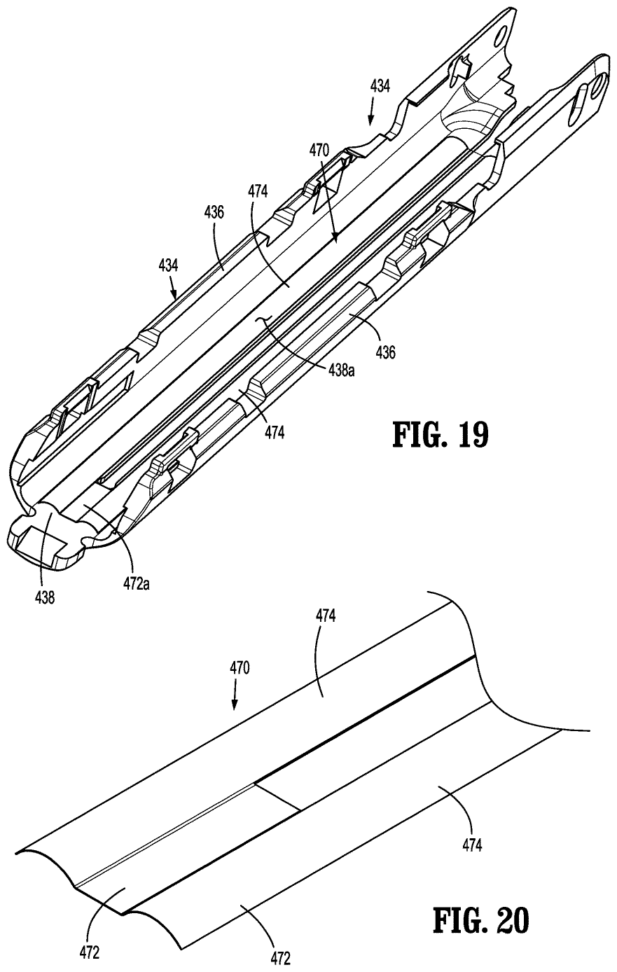

FIGS. 19 and 20 illustrate another version of the channel member and spring shown generally as channel member 434 and spring 470. The channel member 434 is like the channel member 34 (FIG. 4) and includes side walls 436 and a bottom wall 438 that define a cavity 438a that receives the staple cartridge 40 (FIG. 5). The bottom wall 438 supports the spring 470 and defines a knife slot 438a.

The spring 470 includes a base portion 472 and two spaced elongated semi-circular portions 474 that extend proximally from the base portion 472. The semicircular portions 474 are positioned on opposite sides of the knife slot 438a and extend along a substantial portion of the length of the channel member 434. The base portion 472 of spring 470 includes a central portion 472a that is secured to the distal portion of the bottom wall 438 of the channel member 434 by spot welding.

The staple cartridge 40 (FIG. 5) is supported within the cavity 438a of the channel member 434 and rests on the spring 470. When a downward force is applied to the staple cartridge 40, the semicircular portions 474 of the spring 470 flatten to allow the staple cartridge 40 (FIG. 5) to move vertically within the cavity 438a of the channel member 434 to accommodate tissue of different thicknesses.

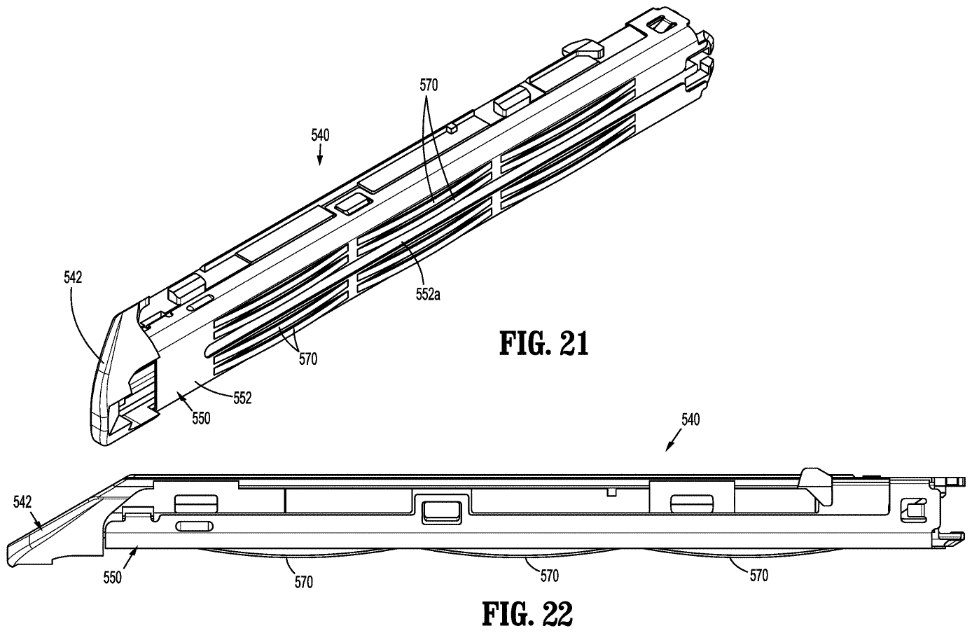

FIGS. 21 and 22 illustrate an alternate version of the staple cartridge shown generally as staple cartridge 540. The staple cartridge 540 is like the staple cartridge 40 (FIG. 5) and includes a cartridge body 542 and a staple guard 550. The cartridge body 542 defines staple receiving slots 56 (FIG. 5) and a knife slot 54 (FIG. 5). The staple guard 550 includes a bottom wall 552 that defines a knife slot 552a that is aligned with the knife slot 54 (FIG. 5) in the cartridge body 542. The bottom wall 552 includes curved spring members 570 that are positioned on opposite sides of the knife slot 54. In aspects of the disclosure, the spring members 570 bow outwardly from the bottom wall 552 of the staple guard 550 along the longitudinal axis of the staple guard 550. In some aspects of the disclosure, the bottom wall 552 of the staple guard 550 includes a plurality of spring members 570 on each side of the knife slot 552a. In certain aspects of the disclosure, the bottom wall 552 of the staple guard 550 includes six spring members 570 on each side of the knife slot 552a and each of the spring members 570 is longitudinally aligned with two other spring members 570.

When the staple cartridge 540 is positioned within the channel member, e.g., channel member 434 (FIG. 19), the spring members 570 rest on the bottom wall 438 of the channel member 434. When a force is applied to the staple cartridge 540, the spring members 570 flatten to allow the staple cartridge 540 to move vertically within the cavity 438a of the channel member 434 to accommodate tissue of different thicknesses.

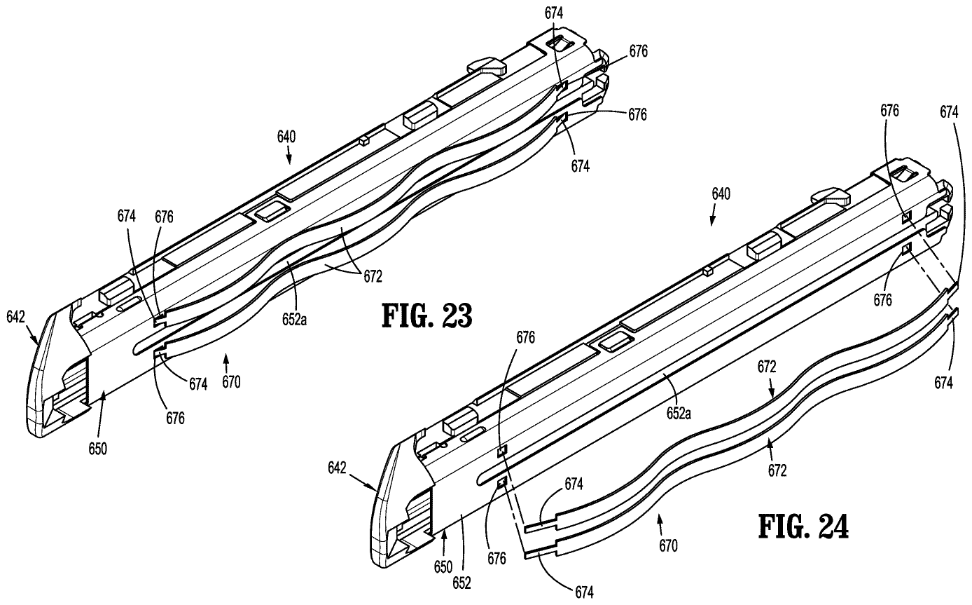

FIGS. 23 and 24 illustrate another version of the staple cartridge shown generally as staple cartridge 640. The staple cartridge 640 is like the staple cartridge 540 (FIG. 21) and includes a cartridge body 642 and a staple guard 650. The cartridge body 642 defines staple receiving slots 56 (FIG. 5) and a knife slot 54 (FIG. 5). The staple guard 650 includes a bottom wall 652 that defines a knife slot 652a that is aligned with the knife slot 54 (FIG. 5) in the cartridge body 642. The staple cartridge 640 differs from the staple cartridge 540 (FIG. 21) in that the staple guard 650 includes spring members 670 that are coupled to the bottom wall 652 of the staple guard 650. In aspects of the disclosure, the spring members 670 are in the form of wave springs 672. Each of the wave springs 672 includes a proximal and a distal extension 674 that are received in openings 676 in the bottom wall 652 of the staple guard 650 to secure the spring members 670 to the staple guard 650. In aspects of the disclosure, the proximal and distal extensions 674 are dimensioned to be slidably received in the openings 676 in the staple guard 650 to allow the wave springs 672 to flatten to allow the staple cartridge 640 to move vertically within the cavity of a channel member, e.g., channel member 434 (FIG. 19), to accommodate tissue of different thicknesses.

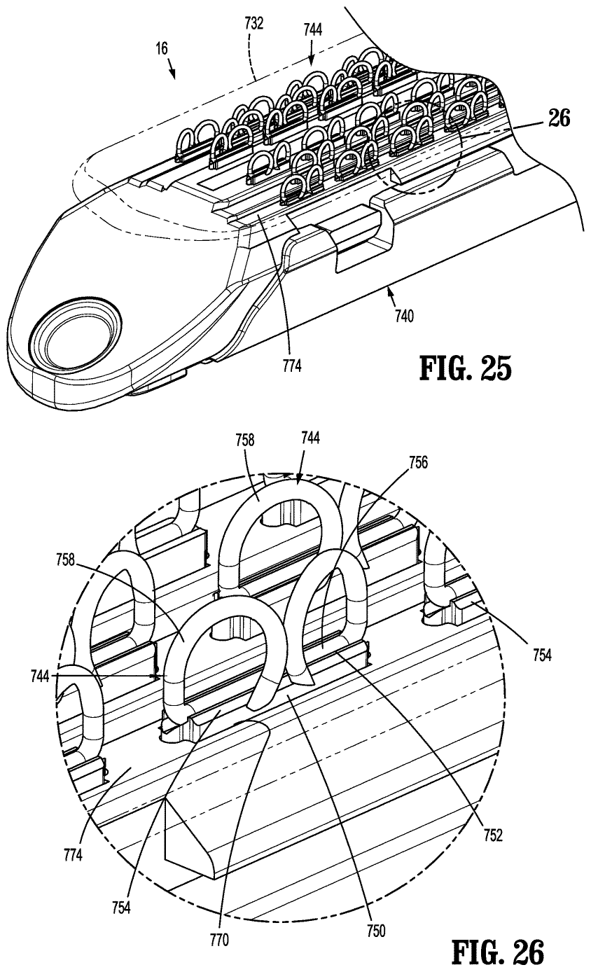

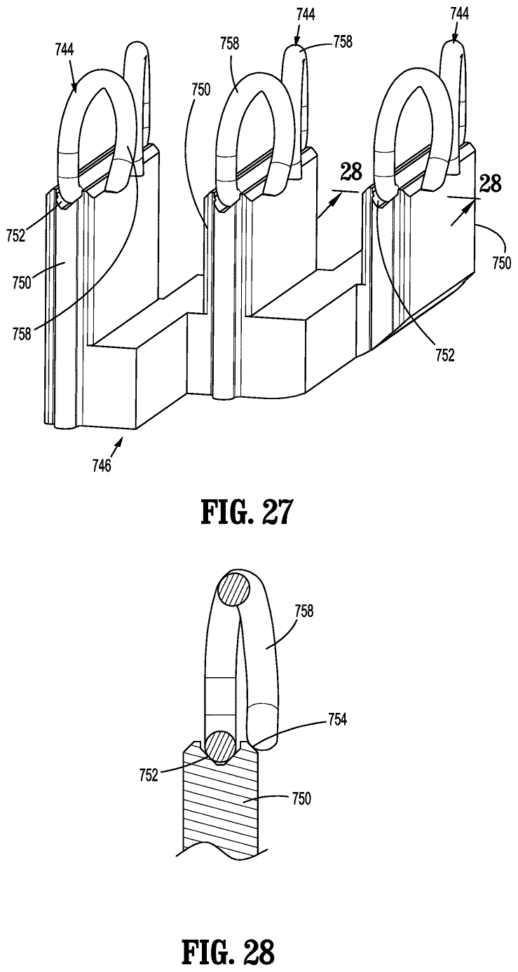

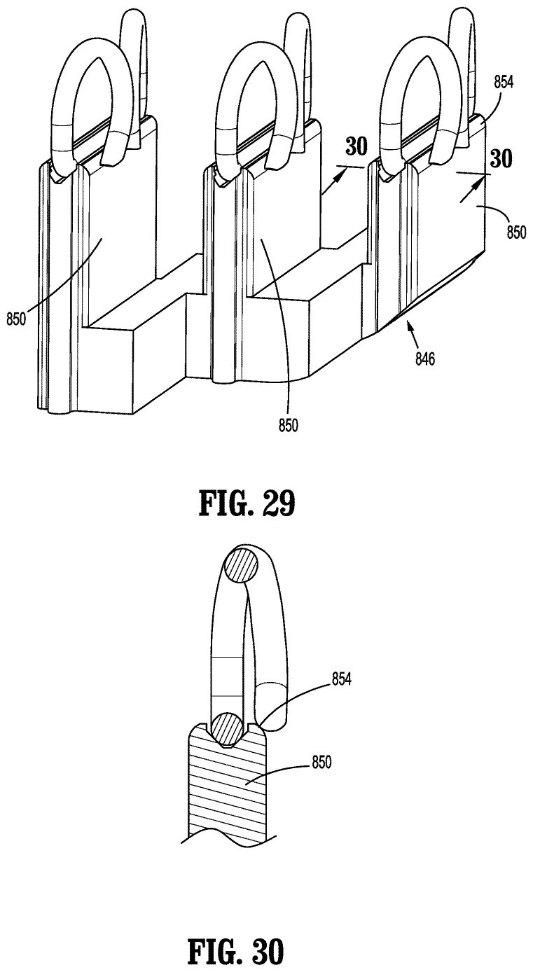

FIGS. 25 to 28 illustrate another version of a staple cartridge shown generally as staple cartridge 740. The staple cartridge 740 includes staples 744 and pushers 746. The pushers 746 are configured to improve staple formation in situations in which the staples 744 may be over crimped. More specifically, each of the staple pushers 746 (FIG. 27) includes one or more pusher plates 750. Each of the pusher plates 746 defines a staple channel 752 that is configured to support a back span 756 of one of the staples 744. The pusher plates 750 also include chamfered surfaces 754 that are positioned along opposite sides of the staple channel 752. The chamfered surfaces 754 are configured to direct legs 758 of a respective one of the staples 744 outwardly of the back span 756 of the staple 744 as the staple 744 is being formed by the anvil 32 (FIG. 1).

FIGS. 25 and 26 illustrate the tool assembly 16 as the tool assembly 16 is fired with the pushers 746. When the stapling device 10 (FIG. 1) is fired, the pushers 746 are pushed through the staple receiving slots 770 of the cartridge body 772 of the staple cartridge 740. As the pushers 746 reach raised positions within the cartridge body 772, the pusher plates 750 extend outwardly beyond a tissue engaging surface 774 of the cartridge body 772 to expose the chamfered surfaces 754 of the pusher plate 750. When the tissue being stapled is thin and the legs 758 of the staple 744 are curled back towards the back span 756 of the staple 744, the legs 758 engage the chamfered surfaces 754 of the pusher plate 750 to direct the legs 758 of the staple 744 outwardly of the back span 756 to prevent malformation of the staple 744.

FIGS. 29 and 30 illustrate an alternate version of a pushers shown generally as pusher 846. The pusher 846 is like the pushers 746 (FIG. 27) except that the chamfered surfaces 754 of the pusher plates 750 are replaced with radiused surfaces 854 on the pusher plates 850. The pushers 846 function in the same manner as the pushers 746 and will not be described in further detail herein.

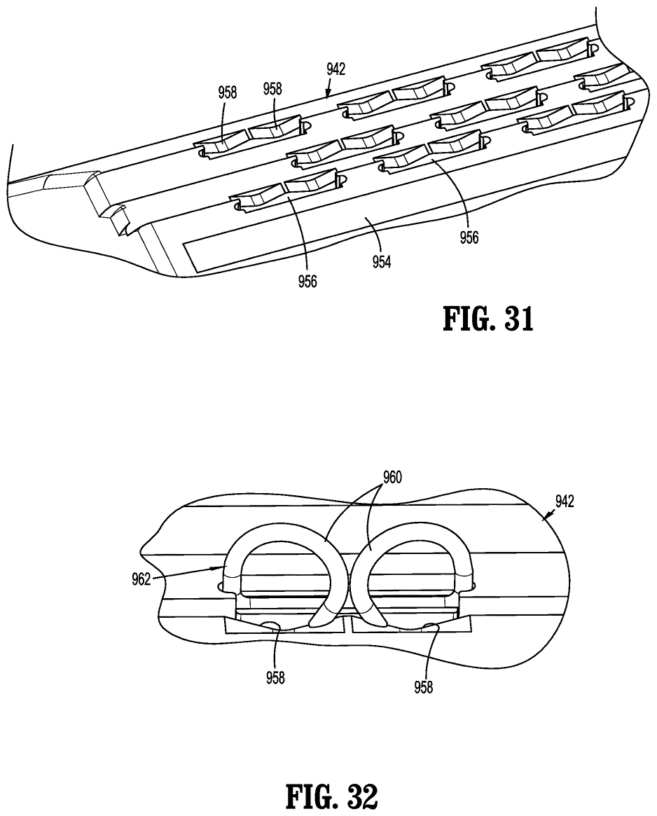

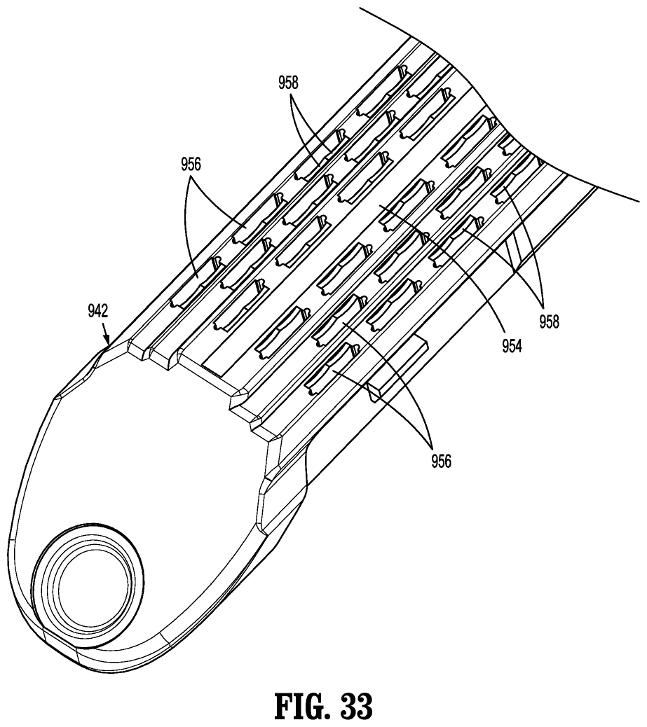

FIGS. 31-33 illustrate an alternate version of the cartridge body of the stapling device of FIG. 1 shown generally as cartridge body 942. The cartridge body 942 defines and a knife slot 954 and staple receiving pockets 956 positioned on each side of the knife slot 954. The cartridge body 942 also defines scalloped pockets 958 that are positioned adjacent to each of the staple receiving pockets 956. Each of the scalloped pockets 958 is configured to curl a leg 960 of a staple 962 upwardly towards the anvil 32 (FIG. 1) to prevent the sharpened tips 960a of the legs 960 of the staples 962 from extending below the back span 964 of the staples 962.

Although each of the aspects of the disclosure are separately described, it is envisioned that each of the aspects of the disclosure can be used with other aspects of the disclosure in a single cartridge assembly. For example, a cartridge assembly having pre-loaded wave springs 170, 270 or 370 can also include the pushers 746 or 846, and/or the cartridge body 942. Similarly, the a cartridge assembly including the spring 470 can also include the pushers 746 or 846, and/or the cartridge body 942.

Persons skilled in the art will understand that the devices and methods specifically described herein and illustrated in the accompanying drawings are non-limiting exemplary aspects of the disclosure. It is envisioned that the elements and features illustrated or described in connection with one exemplary aspects of the disclosure may be combined with the elements and features of another without departing from the scope of the disclosure. As well, one skilled in the art will appreciate further features and advantages of the disclosure based on the above-described aspects of the disclosure. Accordingly, the disclosure is not to be limited by what has been particularly shown and described, except as indicated by the appended claims.

What is claimed is:

1. A cartridge assembly comprising:
a channel member having a proximal portion and a distal portion, the channel member including side walls and a bottom wall that define a cavity, the bottom wall defining a knife slot;
a staple cartridge received within the cavity of the channel member and including a cartridge body, staples, pushers, and a staple guard, the cartridge body defining a knife slot and staple receiving pockets arranged in rows on each side of the knife slot, the staples and the pushers received within the staple receiving pockets, the pushers movable from lowered positions to raised positions within the staple receiving pockets to eject the staples from the staple receiving pockets, the staple guard supported on the cartridge body and positioned to retain the staples and pushers within the cartridge body; and
a spring supported on the channel member between the bottom wall of the channel member and the staple guard of the staple cartridge, the spring secured to the bottom wall of the channel member in a pre-loaded state.

2. The cartridge assembly of claim 1, wherein the channel member includes a distal portion and a proximal portion, the distal portion of the channel member defining an opening that extends through the bottom wall of the channel member.

3. The cartridge assembly of claim 2, wherein the spring includes a wave spring, the wave spring having a distal portion that includes a hook that is received in the opening of the channel member to secure the distal portion of the wave spring to the channel member.

4. The cartridge assembly of claim 3, wherein the wave spring includes a base portion and two elongate spring legs that extend distally from the base portion on opposite side of the knife slot, the base portion supporting the hook.

5. The cartridge assembly of claim 4, wherein each of the two elongate spring legs includes a proximal portion that is secured to the bottom wall of the channel member to secure the wave spring to the channel member in the preloaded state.

6. The cartridge assembly of claim 5, wherein the proximal portion of each of the two elongate spring legs is welded to the bottom wall of the channel member.

7. The cartridge assembly of claim 6, wherein the channel member includes a finger that extends into the opening, and the hook is received about the finger.

8. The cartridge assembly of claim 7, wherein the base portion is formed separately from the two elongate spring legs, and the spring legs are secured to the base portion.

9. The cartridge assembly of claim 5, wherein the opening is dimensioned to facilitate longitudinal movement of the hook within the opening when the wave spring is compressed beyond the pre-loaded state.

10. The cartridge assembly of claim 1, wherein the channel member includes a distal portion and a proximal portion, the distal portion of the channel member defining spaced notches in opposite sides the bottom wall of the channel member.

11. The cartridge assembly of claim 10, wherein the spring includes a wave spring, the wave spring having a distal portion that includes two spaced hooks that are received within the spaced notches of the channel member to secure the distal portion of the wave spring to the channel member.

12. The cartridge assembly of claim 11, wherein the hooks are longitudinally movable within the notches of the channel member when the wave spring is compressed beyond the pre-loaded state.

13. The cartridge assembly of claim 1, wherein each of the pushers includes at least one pusher plate, and each of the staples includes a back span and staple legs that extend from the back span, each of the pusher plates having an upper surface that defines a staple channel that receives the back span of the staple to support the staple on the pusher plate.

14. The cartridge assembly of claim 13, wherein each of the pusher plates includes a radiused surface positioned on each side of the staple channel that is configured to direct the staple legs of a respective one of the staples outwardly of the back span as the staple is being formed.

15. The cartridge assembly of claim 13, wherein each of the pusher plates includes a chamfered surface positioned on each side of the staple channel that is configured to direct the staple legs of a respective one of the staples outwardly of the back span as the staple is being formed.

16. A channel member comprising:
a body having a proximal portion and a distal portion, the body including side walls and a bottom wall that define a cavity, the bottom wall defining a knife slot; and
a spring secured to the bottom wall of the body in a pre-loaded state.

17. The channel member of claim 16, wherein the distal portion of the body of the channel member defines an opening that extends through the bottom wall of the body, and the spring includes a wave spring having a distal portion that includes a hook that is received in the opening of the body of the channel member to secure the distal portion of the wave spring to the body.

18. The channel member of claim 17, wherein the wave spring includes a base portion and two elongate spring legs that extend distally from the base portion on opposite side of the knife slot, the base portion supporting the hook, and each of the two elongate spring legs including a proximal portion that is secured to the bottom wall of the body of the channel member to secure the wave spring to the body of the channel member in the preloaded state.

19. The channel member of claim 16, wherein the distal portion of the body of the channel member defines spaced notches in opposite sides the bottom wall of the body of the channel member, and the spring includes a wave spring having a distal portion that includes two spaced hooks that are received within the spaced notches of the body of the channel member to secure the distal portion of the wave spring to the body of the channel member.

20. A cartridge assembly comprising:
a channel member having a proximal portion and a distal portion, the channel member including side walls and a bottom wall that define a cavity, the bottom wall defining a knife slot;
a staple cartridge received within the cavity of the channel member and including a cartridge body, staples, pushers, and a staple guard, the cartridge body defining a knife slot and staple receiving pockets arranged in rows on each side of the knife slot, the staples and the pushers received within the staple receiving pockets, the pushers movable from lowered positions to raised positions within the staple receiving pockets to eject the staples from the staple receiving pockets, the staple guard supported on the cartridge body and positioned to retain the staples and pushers within the cartridge body; and
a wave spring supported on the channel member between the bottom wall of the channel member and the staple guard of the staple cartridge, the wave spring including a base portion and two spring legs that extend distally from the base portion along opposite sides of the knife slot, the two spring legs secured to the bottom wall of the channel member in a pre-loaded state.
